(19) United States
(12) Patent Application Publication
(10) Pub. No.: US 2010/0055797 A1
(43) Pub. Date: Mar. 4, 2010

(54) Nano-construction processes and analytical processes

(76) Inventor: Michael Reading, Norfolk (GB)

Correspondence Address: SENNIGER POWERS LLP, 100 NORTH BROADWAY, 17TH FLOOR, ST LOUIS, MO, 63102, US

(21) Appl. No.: 11/920,203

(22) PCT Filed: May 9, 2006
(86) PCT No.: PCT/GB2006/001689

§ 371 Date: Nov. 5, 2009

(30) Foreign Application Priority Data
May 9, 2005 (GB) 0509427.1

(51) Int. Cl. G01N 33/50; C07K 14/00; C07H 21/02; C07H 21/04

(52) U.S. Cl. 436/86; 530/350; 536/23.1; 436/94

(57) ABSTRACT

A process for assembling molecules on a surface, the process comprising: providing a probe having a variable-temperature tip; providing a first material, which is optionally on a first surface of a substrate; bringing the tip of the probe into contact with the first material, whereby molecules of the first material are transferred to the tip; and moving the tip of the probe to a position above a surface of a second material (the second surface); then heating the tip of the probe to a temperature T2, at which the first material will transfer from the tip to the surface of the second material. Analytical process using this method are also disclosed.

The present invention relates to nano-construction processes for assembling molecules on a surface, which may subsequently react with the surface, and analytical techniques, which may be used in combination with the nano-construction processes.

Recent advances in atomic force microscopy have allowed the transfer of small quantities of material from one place to another using heated probes as first described in U.S. Pat. No. 6,405,137. In this way material may be transferred to a surface using a small tip. Such a process is termed nanolithography and is illustrated in a paper by Sheehan et al in Applied Physics Letters, Volume 85, No. 9, page 1589-1591, published on 30 Aug. 2004. In a process described in this paper, a tip of an atomic force microscope is coated with two monolayers of octadecylphosphonic acid (OPA) by evaporating the OPA onto the tip. The tip is then positioned above a mica surface and heated to a temperature above the melting point of the OPA, at which point the OPA is deposited onto the surface. The process of initially evaporating a substance to be transferred onto the tip is a time consuming procedure, which must be carried out carefully and with the correct equipment at the correct temperature, i.e. a temperature which ensures evaporation of, but not degradation of, the evaporating substance. The process of evaporating the OPA may not be suitable for all materials, which for example, do not evaporate easily or are liable to degrade at a temperature near the evaporation temperature. There may also be a degree of wastage in evaporating the material onto the tip, since not all of the material evaporated will condense onto the tip.

The present invention aims to mitigate or overcome at least some of the problems associated with the prior art and describes new methods of manipulating materials on surfaces so that a wide variety of structures can be achieved.

In a first aspect, the present invention provides a process for assembling molecules on a surface, the process comprising:

providing a probe having a variable-temperature tip;

providing a first material, which is optionally on a first surface of a substrate;

bringing the tip of the probe into contact with the first material, whereby molecules of the first material are transferred to the tip; and

moving the tip of the probe to a position above a surface of a second material (the second surface); then

heating the tip of the probe to a temperature T2, at which the first material will flow from the tip to the surface of the second material. The molecules of the first material preferably contact the second surface prior to or during the heating of the tip to temperature T2.

The first material may have a melting or softening point Tm, which is preferably above the ambient temperature of the device. Preferably, during transfer of the first material to the probe, the temperature of the first material is at or above its melting or softening point, Tm. This may be effected by heating the tip either before contacting or while in contact with the first material to a temperature T1, which is preferably at or above Tm. Alternatively, the first material may be heated prior to contact with the probe such that it is at or above Tm.

Preferably, once the first material has transferred to the tip, if the tip is not below Tm, then the tip is cooled to a temperature Tc, which is below Tm. The tip may be cooled either while still in contact with the first material while being moved to a position above the second surface.

The tip of the probe may be a tip of a probe of an atomic force microscope known to those in the art. Preferably, the tip has a radius of curvature, or a ‘sharpness’, of 200 nm or less, preferably 100 nm or less. The tip preferably has a sharpness of less than 50 nm.

The probe may comprise silicon, which can be fabricated with a standard silicon-on-oxide cantilever fabrication process. Such tips are further described in a paper by Sheehan et al in Applied Physics Letters, Volume 85, No. 9, page 1589-1591, published on 30 Aug. 2004. Additionally, see references 8 to 12 listed in column 2 of page 1591 of the Sheehan paper. The probe may have a heating time of 1 to 50 μs, preferably 1 to 20 μs. The probe may have a cooling time of 1 to 100 μs, preferably 1 to 50 μs. The probe may be capable of being heated to temperature of 700° C., preferably 1000° C.

The probe may be a probe of a scanning thermal microscope. The probe may comprise a loop of Wollaston wire, which may be shaped in the form of a cantilever, the end of which forms a resistive element. The probe may be a platinum/rhodium resistance thermal probe. Such probes are further described in U.S. Pat. Nos. 6,095,679; 6,200,022, 6,405,137 and 6,491,425. The probe and the microscope may be as described in any one of these documents.

Preferably, the first material is solid at the ambient temperature of the device which may be controlled by locating it within a cooling or heating chamber. The softening and/or melting temperatures of the materials being used would typically be 10 or more degrees Celsius above the ambient temperature.

The substrate and second material may comprise the same or different materials. The substrate and/or the second material may comprise a metal, to which molecular moieties may be bound, or an organic substance, such as paracetamol.

The process may be applied to transfer any material using the probe. The first materials may comprise any material such as, for example, a synthetic polymer, such as polyethylene or polyethyleneglycol, or a natural polymer, such as DNA, or a pharmaceutical such as paracetamol. For example, the first material may comprise compounds such as individual nucleotides of DNA and RNA, i.e. monophosphates of adenine (adenosine 3′-monophosphate), cytosine (cytidine 3′-monophosphate), guanine (guanosine 3′-monophosphate), thymine (thymidine 3′-monophosphate) and uracil. These compounds are particularly preferred if it is desired to construct a DNA or RNA chain, as described below. The first material may comprise amino acids or peptides.

An example first material comprising an organic compound is octadecylphosphonic acid (OPA). This compound has a melting point of 99° C. A sample of the OPA-material could be transferred from one position to another by placing a tip of a probe above the surface of OPA (the OPA being at room temperature), raising the temperature of the tip to 110° C. contacting the tip with the surface of the OPA and allowing a sample of the OPA to transfer to the tip. The tip could then be cooled below 99° C., e.g. to a temperature of 20° C., raised and moved to a position above a second surface. The temperature of the tip could then be raised to 110° C., allowing the OPA to flow from the tip to the second surface.

T2 is a temperature preferably at or above the melting point of the first material to allow sufficiently fast flow of the first material from the tip to the second surface.

Reactive moieties may be present on the second surface, which will react with, and preferably form a chemical bond with, the molecules of the first material being transferred. Such reactive moieties may, for example, be organic compounds bound to the surface of the second material, for example the species bound to the surface may comprise a free isocyanate group and the molecules of the material deposited by the probe may each have a hydroxyl group and under the suitable action of temperature, they could form a covalent bond. Other groups that may be present on the surface may comprise the very wide range of reactive groups well known to those skilled in the art of synthetic organic chemistry. The first material is preferably selected such that covalent bonds can form between the free reactive groups of the material on the second surface and the molecules of the first material.

The process may comprise the step of providing a second material having reactive moieties on its surface, where said moieties will form a chemical bond with the first and/or further material being transferred. Such a process will be referred to as a ‘construction process’ from hereon. When the first material transfers from the tip to the second surface, each reactive moiety on the second surface reacts and forms a chemical bond with a molecule of the first material. The chemical bond is preferably a covalent bond, but may be a hydrogen bond. Preferably, subsequent to the formation of the chemical bonds between the transferred material and the reactive moieties, the surface is flushed with a solvent, which will remove any material that is not bound to the reactive moieties. The solvent may act to cleave any bonds the first material has made with the surface, but will not act to break bonds formed between the reactive moieties and the first material. Suitable solvents for removing the non-bonded chemical moieties include, but are not limited to, water or common organic solvents such as acetone. If the resultant species formed from reacting the transferred first material with the reactive moieties is in itself reactive, this may act as a second reactive moiety, suitable for bonding to further material transferred to the second surface. The further material may the same as or different from the first material.

The present invention further provides a process including a repeated sequence, where the sequence comprises: (i) transfer of a selected material from a first surface to a second surface using the process of the first aspect described above, (ii) reaction of molecules of the transferred material with reactive moieties present on the second surface to form further reactive moieties, and (iii) optionally flushing the surface with a solvent to remove molecules of the first material on the second surface which are not bound to the reactive moieties initially present on the second surface, can be employed in order to build chains of molecules attached to the surface having a desired sequence.

As an example of the repeated-sequence process, the reactive moiety may be a thiol bound to the surface of a suitable substrate, where the sulphur atom of the thiol which is attached via a hydrocarbon chain to, for example, a reactive ester (e.g. (surface)-S—(CH₂)ₙ—(C═O)—O—(C═O)—R, wherein n is from 1 to 10 and R is a C₁-C₁₀ alkyl group). The first material being transferred may comprise an individual nucleotide base such as cytidine 3′-monophosphate. The cytidine 3′-monophosphate is transferred using the process of the first aspect as described above to the second surface, upon which are reactive ester groups. The phosphate group of the cytidine 3′-monophosphate then bonds to the ester group. The surface is then washed using an inert solvent such as suitably buffered water. A second material is then transferred using the same process, the second material comprising thymidine 3′-monophosphate. The phosphate of the thymidine 3′-monophosphate will react with the 5′-hydroxy group of the cytidine 3′-monophosphate, which is attached to the second surface via the ester groups. Repeating this procedure would construct a DNA molecule attached to the surface having the sequence:

(surface) -C-T-C-T-C-.

The process could be applied to create any desired sequence of DNA or RNA. The DNA and RNA could be separated from the surface and then multiple copies of the DNA/RNA sequence could be produced by using methods known to those skilled in the art, such as PCR. Protecting groups and/or catalysts may be used, as would be well known to the person skilled in the art.

In a second aspect, the present invention further provides a method for controlling the location of chemical bond formation on a surface, the process comprising:

providing a surface having first reactive moieties associated therewith and second reactive moieties in contact with the first reactive moieties, wherein the second reactive moieties are capable of forming chemical bonds at a significant rate with the first reactive moieties above a temperature T_{A}, said surface being at a temperature below temperature T_{A},

bringing a probe having a temperature of at least T_{A} into contact with the first and second reactive moieties, such that the first reactive moieties form chemical bonds with the second reactive moieties. For example one end of a hydrocarbon chain (preferably a C₁ to C₂₀ hydrocarbon chain) may be bound to the first surface via a thiol group while having, unbound, at the other end, an isocyanate group (e.g. (surface-S—C₁₋₂₀ alkyl chain-N≡C). Located on or around these reactive moieties may be a solution containing polymer chains having hydroxyl groups. The probe, close to or touching the surface is raised to or slightly above T_{A}.

Reaction between the hydroxyl groups and the isocyanate groups would occur in the immediate vicinity of the probe, but not elsewhere, thus binding the hydroxyl functionalised polymer chains to selected location on the surface.

“Significant rate” includes, but is not limited to a rate more than 10 times faster, preferably more than 100 times faster, more preferably more than 10ⁿ times faster, wherein n is 3 to 6, than the equivalent reaction at the ambient temperature of the second surface.

The probe may be as described above in relation to the first aspect of the invention.

The first reactive moieties may have been transferred to the surface by the process defined above in the first aspect of the invention.

The present invention further provides the following analytical processes.

### Analytical Process 1

The present invention provides a first analytical process comprising:

providing a probe having a tip,

providing a material comprising probe molecules,

bringing the tip of the probe into contact with the material comprising probe molecules, whereby probe molecules are transferred to the tip,

providing a surface having target molecules thereon,

bringing the tip of the probe into close proximity or contact with the target molecules so that the target and probe molecules interact,

and detecting the interactions between the probe molecules and the target molecules, optionally by one of the methods defined in f. below. The process may further comprise the step of forming the surface having target molecules thereon by either (i) transferring the target molecules to the surface using the process of the first aspect of the invention or (ii) using the process of the second aspect of the invention to construct the target molecules on the surface from reactive moieties.

In a preferred aspect, the first analytical process comprises the following steps:
a) The thermal probe is first cleaned by heating it to a high temperature.
b) (Optional) a suitable coating is caused to form on the thermal tip by placing it in suitable liquid or a solid that is rendered fluid by heating with the probe.
c) Probe molecules, i.e. molecule used to probe the properties of the target molecules discussed below, are attached to the tip by placing it adjacent to the molecules on a surface in such a way as to cause the probe molecules to transfer to the tip (i.e. because it will be attracted to or bound to the coating, if present) or the tip is placed in a liquid containing the probe molecules or onto a solid that contains probe molecules, the solid being rendered fluid by local heating.
d) Target molecules are distributed onto a surface possibly solvated by a thin layer of solvent.
e) The tip with the probe molecule(s) attached is placed on the surface or into the solution in such a way as to present the probe molecule to the target molecules so that they have an opportunity to interact.
f) Interactions are detected by either one of more than one of the following measurements
   i) The tip is withdrawn and the force required to detach the tip from the surface indicates whether an interaction has occurred.
   ii) A temperature change can be detected by the tip and this calorimetric signal can indicate whether an interaction has occurred.
   iii) The temperature of the tip can be programmed and so a local scanning calorimetry experiment, with or without temperature modulation, combined with thermo mechanical and/or dynamic thermo mechanical analysis can detect whether an interactions has occurred possibly by detecting a transition temperature or a change in transition temperature
   iv) The tip can be irradiated with electromagnetic radiation at a suitable frequency or frequencies and this photo thermal spectroscopy measurement can indicate whether an interaction has occurred.
   v) The tip can be heated to a high temperature causing the molecules on its surface to volatilise or decompose, these volatised and decomposed species then being transported by a flow of air into a mass spectrometer and then analysed.
      The analytical methods are described in f) above are described in: U.S. Pat. No. 6,095,679, U.S. Pat. No. 6,405,137, U.S. Pat. No. 6,200,022, U.S. Pat. No. 6,491,425 and the papers of Reading, et al, e.g.: ‘Thermally assisted nanosampling and analysis using micro-IR spectroscopy and other analytical methods’, published in Vibrational Spectroscopy, 29, (2002) 257-260, also in Hammiche A., Bozec L., German M. J., Chalmers J. M., Everall N. J., Poulter G., Reading M., Grandy D. B., Martin F. L. and Pollock H. M., ‘Mid-infrared micro-spectroscopy of difficult and awkward samples: near field photothermal micro-spectroscopy (PTMS), a novel, non-destructive microprobe approach’, published in Spectroscopy 19(2), 20-42 (February 2004) with erratum in 19(5), 14 (2004), also in Reading M., Price D. M., Grandy D. B., Smith R. M., Bozec L., Conroy M., Hammiche A. and Pollock M. P., ‘Micro-thermal analysis of polymers: current capabilities and future prospects’, published in Macromol. Symp., 167, 2001, 45-62, and also in Grandy, D. B.; Hourston, D. J.; Price, D. M.; Reading, M.; Goulart Silva, G.; Song, M.; Sykes, P. A.; ‘Micro-thermal characterization of segmented polyurethane elastomers and a polystyrene-poly(methyl methacrylate) polymer blend using variable-temperature pulsed force mode atomic force microscopy’, published in Macromolecules 33, 2000, 9348-9359.

### Analytical Process 2

The present invention provides a second analytical process comprising:

providing a probe having a tip,

providing a material comprising target molecules,

bringing the tip of the probe into contact with the material comprising target molecules, whereby target molecules are transferred to the tip,

providing a surface having probe molecules thereon,

bringing the tip of the probe into close proximity or contact with the probe molecules so that the target and probe molecules interact,

and detecting the interactions between the probe molecules and the target molecules, optionally by one of the methods defined in f. below. The process may further comprise the step of forming the surface having probe molecules thereon by either (i) transferring the probe molecules to the surface using the process of the first aspect of the invention or (ii) using the process of the second aspect of the invention to construct the probe molecules on the surface from reactive moieties.

In a preferred aspect, the second analytical process comprises the following steps:
a) Probe molecules are distributed onto a surface, which may be by synthesising these molecules in situ on the surface using the construction process described above. These molecules might possibly be solvated by a thin layer of solvent.
b) The tip is cleaned by heating it to a high temperature.
c) (Optional) a suitable coating is caused to form on the thermal tip by placing it in suitable liquid or a solid that is rendered fluid by heating with the probe.
d) Target molecules are attached to the tip by placing the tip adjacent to the target molecules on a surface is such a way as to cause the target molecules to transfer to the tip (i.e. because it will be attracted to or bound to the coating) or the tip is placed in a liquid comprising the target molecules or molecules or onto a solid that comprises the target molecules and, where the tip is rendered fluid by local heating.
e) The tip with the target molecule(s) attached is placed on the surface or into the solution in such a way as to present the target molecule to the probe molecules so that they have an opportunity to interact.
f) Interactions are detected by either one or more than one of the following measurements
   a. The tip is withdrawn and the force required to detach the tip from the surface indicates whether an interaction has occurred.
   b. A temperature change can be detected by the tip and this calorimetric signal can indicate whether an interaction has occurred.
   c. The temperature of the tip can be programmed and so a local scanning calorimetry experiment, with or without temperature modulation, combined with thermo mechanical and/or dynamic thermo mechanical analysis can detect whether an interactions has occurred possibly by detecting a transition temperature or a change in transition temperature
   d. The tip can be irradiated with electromagnetic radiation at a suitable frequency or frequencies and this photo thermal spectroscopy measurement can indicate whether an interaction has occurred.
   e. The tip can be heated to a high temperature causing the molecules on its surface to volatilise or decompose, these volatised and decomposed species then being transported by a flow of air into a mass spectrometer and then analysed.
      The analytical methods are described in f) above are described in: U.S. Pat. No. 6,095,679, U.S. Pat. No. 6,405,137, U.S. Pat. No. 6,200,022, U.S. Pat. No. 6,491,425 and the papers of Reading, et al, e.g: ‘Thermally assisted nanosampling and analysis using micro-IR spectroscopy and other analytical methods’, published in Vibrational Spectroscopy, 29, (2002) 257-260, also in Hammiche A., Bozec L., German M. J., Chalmers J. M., Everall N. J., Poulter G., Reading M., Grandy D. B., Martin F. L. and Pollock H. M., ‘Mid-infrared micro-spectroscopy of difficult and awkward samples: near field photothermal micro-spectroscopy (PTMS), a novel, non-destructive microprobe approach’, published in Spectroscopy 19(2), 20-42 (February 2004) with erratum in 19(5), 14 (2004), also in Reading M., Price D. M., Grandy D. B., Smith R. M., Bozec L., Conroy M., Hammiche A. and Pollock M. P., ‘Micro-thermal analysis of polymers: current capabilities and future prospects’, published in Macromol. Symp., 167, 2001, 45-62, and also in Grandy, D. B.; Hourston, D. J.; Price, D. M.; Reading, M.; Goulart Silva, G.; Song, M.; Sykes, P. A.; ‘Micro-thermal characterization of segmented polyurethane elastomers and a polystyrene-poly(methyl methacrylate) polymer blend using variable-temperature pulsed force mode atomic force microscopy’, published in Macromolecules 33, 2000, 9348-9359.

### Analytical Process 3

The present invention provides a third analytical process comprising

a) either
i) providing a surface having thereon probe molecules and transferring target molecules to the surface using the process of the first aspect of the invention (in which the target molecules are molecules of the first material), or
ii) providing a surface having thereon target molecules and transferring probe molecules to the surface using the process of the first aspect of the invention (in which the probe molecules are molecules of the first material)

and b) detecting interactions between the target molecules and the probe molecules, optionally by any of the techniques described in c) below.

In a preferred aspect, the third analytical process comprises the following steps:
a) Probe molecules are distributed onto a surface, which may be by synthesising these molecules in situ using the construction process described above. These molecules might possibly be solvated by a thin layer of solvent.
b) Target molecules are introduced to the probe molecules by flushing the surface by micro-fluidics or a similar system then non-bound molecules are flushed away leaving behind certain target molecules bound to certain probe molecules.
c) The probe molecules that have interacted with target molecules are now detected by one or more of the following methods;
   i) The surface is imaged by the probe using a contact or intermitted contact or non-contact method and changes due to the bonded target molecules are seen as changes in the corresponding images.
   ii) The tip is placed on a site know to contain a probe molecule and whether or not it has interacted will be determined by scanning the temperature of the tip so a local scanning calorimetry experiment, with or without temperature modulation, combined with thermo mechanical and/or dynamic thermo mechanical analysis can detect whether an interactions has occurred possibly by detecting a transition temperature or a change in transition temperature
   iii) The tip is placed on a site know to contain a probe molecule and whether or not it has interacted will be determined by irradiating the tip with electromagnetic radiation at a suitable frequency or frequencies and this photo thermal spectroscopy measurement can indicate whether an interaction has occurred.
   iv) The tip is placed on a site know to contain a probe molecule and whether or not it has interacted will be determined by heating the tip to a high temperature causing the molecules on the surface to volatilise or decompose, these volatised and decomposed species then being transported by a flow of air into a mass spectrometer and then analysed.
      The analytical methods are described in f) above are described in: U.S. Pat. No. 6,095,679, U.S. Pat. No. 6,405,137, U.S. Pat. No. 6,200,022, U.S. Pat. No. 6,491,425 and the papers of Reading, et al, e.g: ‘Thermally assisted nanosampling and analysis using micro-IR spectroscopy and other analytical methods’, published in Vibrational Spectroscopy, 29, (2002) 257-260, also in Hammiche A., Bozec L., German M. J., Chalmers J. M., Everall N. J., Poulter G., Reading M., Grandy D. B., Martin F. L. and Pollock H. M., ‘Mid-infrared micro-spectroscopy of difficult and awkward samples: near field photothermal micro-spectroscopy (PTMS), a novel, non-destructive microprobe approach’, published in Spectroscopy 19(2), 20-42 (February 2004) with erratum in 19(5), 14 (2004), also in Reading M., Price D. M., Grandy D. B., Smith R. M., Bozec L., Conroy M., Hammiche A. and Pollock M. P., ‘Micro-thermal analysis of polymers: current capabilities and future prospects’, published in Macromol. Symp., 167, 2001, 45-62, and also in Grandy, D. B.; Hourston, D. J.; Price, D. M.; Reading, M.; Goulart Silva, G.; Song, M.; Sykes, P. A.; ‘Micro-thermal characterization of segmented polyurethane elastomers and a polystyrene-poly(methyl methacrylate) polymer blend using variable-temperature pulsed force mode atomic force microscopy’, published in Macromolecules 33, 2000, 9348-9359.

### Analytical Process 4

This process described below may be used to carry out electrophoresis on small scale (of the order of tens of microns or less) not previously possible.

The present invention provides a fourth analytical process comprising:

providing a substrate having thereon a surface coating, wherein the surface coating has at least one property which varies in a direction across the surface,

placing a sample on the surface coating,

and separating the components of the sample along the said direction and detecting and identifying the components of the sample. The fourth analytical process may further comprise the step of forming the surface coating using the processes of the first or second aspects of the present invention.

In a preferred aspect, the fourth analytical process comprises the following steps:
a) An appropriate surface coating on a substrate, the coating comprising molecules attached to reactive moieties on the surface is prepared using the construction process described above or the process of the second aspect of the invention. The surface coating has been constructed such that across the surface the properties of the coating molecules changes, preferably in a gradual manner. For example, the pH of the molecules of the surface coating may vary gradually, i.e. increasing in one particular direction. The pH gradient could, for example, be formed by using a the process of the second aspect of the invention as described above, for example a network of hydroxyl functional polymer chains could be attached to part of the surface using the second aspect of the invention described on page 8 above. The surface could then be exposed to a solution of an oxidising agent, for example potassium permanganate, at a temperature at which no significant reaction occurred. Local heating with the probe would enable local oxidation of the hydroxyl groups to acid groups. By heating some areas for longer or at a higher temperature than others the concentration of acid groups formed on the polymer chains in some locations would be higher than others. The surface could then be purged of the oxidising agent leaving behind some areas where the local pH was higher than other in a controlled way. This is just one example of how local variations in pH could be achieved over a surface there are many alternative combinations of reagents that would achieve the same effect and these are known to those skilled in the art of synthetic organic chemistry. For example, a strip of coating, with a pH gradient across the coating could be formed next to an ultra-thin square of suitable gel material commonly used in electrophoresis.
b) The molecules of the coating may then be solvated, for instance by controlling the vapour pressure of a suitable solvent and the temperature of the surface onto which the film is deposited or by micro fluidic devices flooding the surface.
c) The sample to be analysed is placed using a tip or by some other process onto an appropriate point on the surface coating.
d) An electric potential is then applied across the coating in the direction of the gradually changing properties. The electric potential across the surface may, for example, be created either by using electrodes that pre-existed on the surface or by using the tip, which transferred the sample to the surface, in combination with another tip or an electrode already on the surface, in order to effect electrophoresis. If the property which varied across the coating was pH, isoelectric focusing could be carried out such that the components of the sample separate and each component comes to rest at a point on the surface having a particular pH, at which the isoelectric point of that component is zero (i.e. the component's net charge is then zero at that particular point). Separation by isoelectric focussing is particularly useful when the sample contains a mixture of proteins, DNA, RNA, or other macromolecules.
e) (Optional) a second electrophoresis could be carried out. For example, the components already separated by isoelectric focusing could be further separated through the adjacent gel film in a direction substantially perpendicular to the first separation direction. Prior to the second electrophoresis being carried out, the components separated in the first step could be reacted with reagents delivered to the surface using the process of the first aspect and/or second aspect of the invention or simply by flooding the surface with the reagent. For example the reagent could be sodium dodecylsulphate (sometimes termed SDS) which is commonly used after isoelectric focusing and prior to a second separation step. SDS is an anionic detergent that disrupts non-covalent interactions in proteins.
f) (Optional) the solvent could be removed by volatilisation, which may involve flushing the surface with a suitable low boiling point solvent.
g) (Optional) the material on the surface could be exposed to a reagent or stain that will facilitate their detection and/or identification and/or quantification. Such reagents for staining molecules such as RNA, DNA and proteins are known to those skilled in the art.
h) The resultant chromatogram (i.e. the substrate having the separated components on its surface, which resulted from the first and, possibly, the optional second electrophoresis) could be analysed with a tip of a suitable microscope (for instance, the thermal tip of a scanning thermal microscope) to obtain images of topography, thermal, photothermal and/or mechanical properties. Once positions where the separated components are located on the chromatogram have been identified, they could be analysed by placing the tip at selected locations and using one or more of the following;
   a. The tip is placed on a selected site and an analysis is carried out by a local scanning calorimetry experiment, with or without temperature modulation, combined with thermo mechanical and/or dynamic thermo mechanical analysis.
   b. The tip is placed on a selected site and an analysis is carried out by irradiating the tip with electromagnetic radiation at a suitable frequency or frequencies thus enabling photo thermal spectroscopy.
   c. The tip is placed on a selected site and an analysis is carried out by heating the tip to a high temperature causing the molecules on the surface to volatilise or decompose, these volatised and decomposed species then being transported by a flow of air into a mass spectrometer and then analysed.
      The methods of analysis a., b. and c. are known to those skilled in the art.
      The present invention will now be described by way of example only with reference to the following drawings, in which

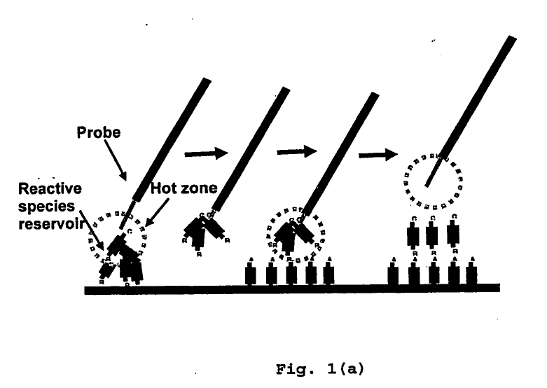

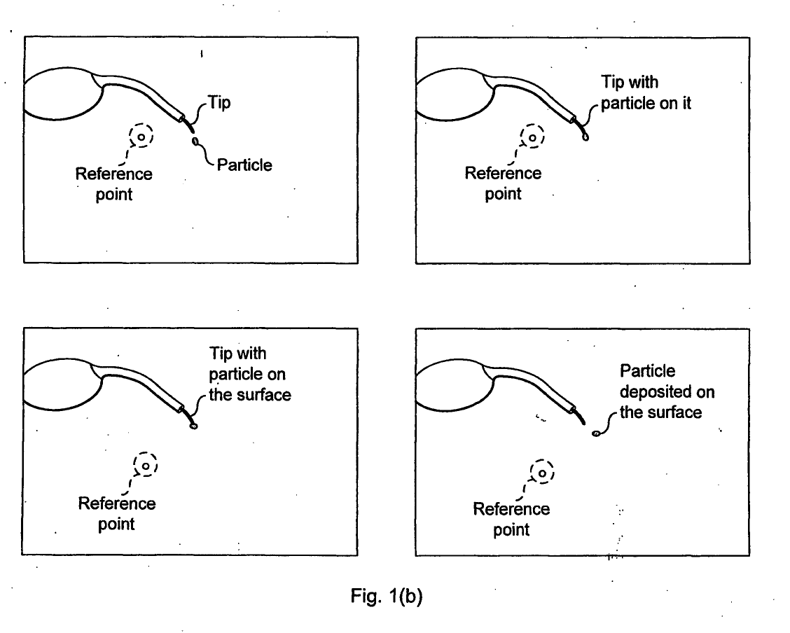

FIG. 1(a) shows an illustration of an embodiment of the first aspect of the present invention, in which a heated tip of a probe is shown to first contact in the left hand diagram a first material (‘reactive species’) on a first surface, such that a sample of the first material adheres to the tip. In the second diagram, the tip having the sample of the first material thereon is cooled and removed from the main body of the first material. In the third diagram, the probe is brought to a position above a second surface, whereby reactive moieties are provided on the second surface. The tip is then heated such that the sample of the first material is transferred to the second surface. The molecules of the first material react and form bonds with the reactive moieties, as shown in the fourth (right hand) diagram. The tip is removed from the second surface.

FIG. 1 (b) shows a series of photographs illustrating how material can be picked up and deposited onto a surface in the way shown in FIG. 1(a) (note that a reference position is marked by a red circle); in the first photograph the tip is above a particle on a surface, in the second photograph, the heated tip has been placed on the particle and raised so that the particle is now located on the tip, in the third photograph the heated tip, with the particle attached, is placed on the surface, in the final photograph it can be seen that the particle has been deposited onto the surface.

FIG. 1 (c) shows the photothermal infrared spectra obtained using the same tip that manipulates the particle shown in the FIG. 1 (b)—from polyethyleneglycol (PEG) as the red spectrum (line A), from paracetamol as the blue spectrum (line B) and a black spectrum (line C) which is the spectrum obtained when a tip holding a PEG particle is placed on a paracetamol surface. The green arrows indicate where the spectral features from the paracetamol appear in the black spectrum in addition to the PEG spectrum thus both materials can be seen. In ways well known to those skilled in the art, a reaction or some other form of interaction between them can be detected by changes in features in such a spectrum.

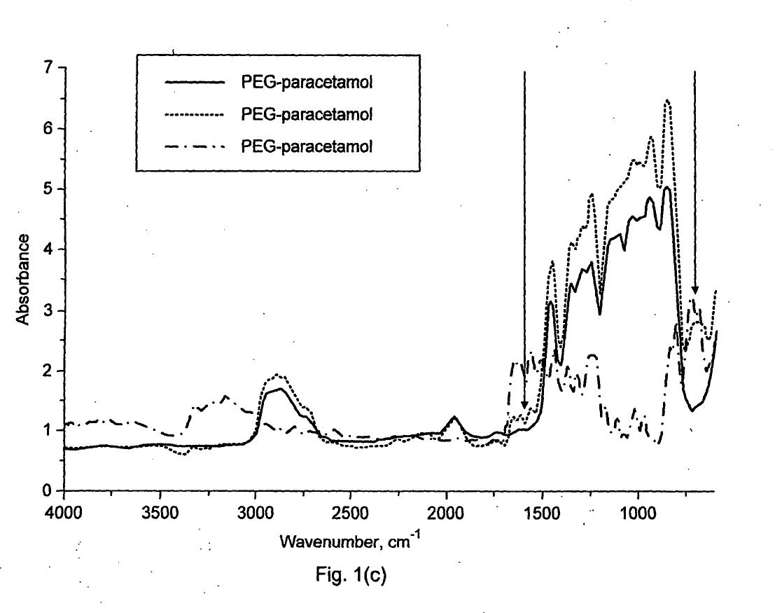

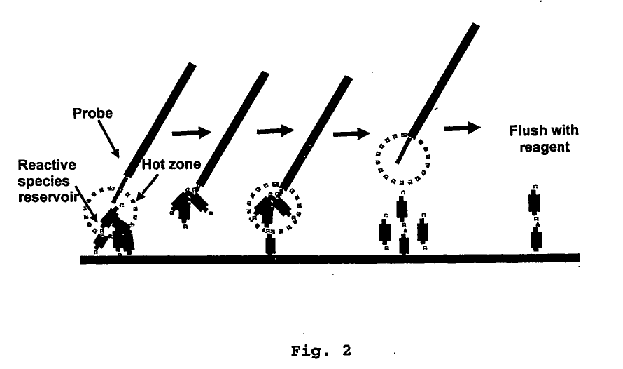

FIG. 2 shows an illustration of an embodiment, substantially the same as in FIG. 1(a), with the exception that an excess of the first material is transferred, such that not all of the first material can bond with the reactive moieties on the second surface. The remaining first material on the second surface is removed by a solvent or reagent such that all molecules of the first material are removed from the second surface, except if they have formed a bond with the reactive moieties.

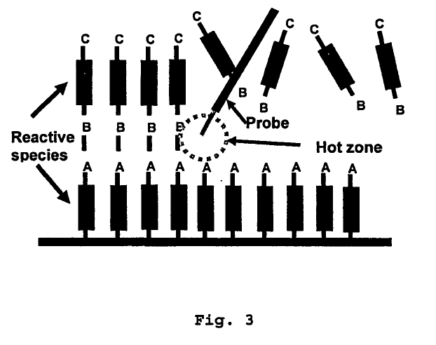

FIG. 3 illustrates the second aspect of the invention and shows the heated tip of a probe being brought into proximity to a surface having reactive moieties thereon and a first material which will react and forms bonds with the reactive moieties when the heat of the probe is sufficiently high.

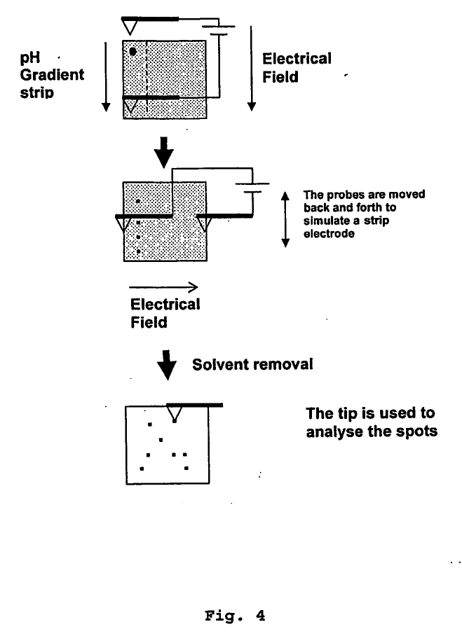

FIG. 4 illustrates a 2D electrophoresis on a gel, whereby in the top diagram, a spot of a sample (which contains a mixture of components) is shown on a pH gradient strip, the pH varying from top to bottom and two tips of a probe which act as electrodes to create an electric potential in the direction of the varying pH gradient. The second, middle diagram shows the result of applying the electric gradient, i.e. the sample has separated into a number of components, each settling at a pH on the surface at their isoelectric point. The tips of the probes are shown to be moved such that a second electric potential is created at approximately 90° to the first electric potential to carry out a second electrophoresis (this time separating the components by weight). The tips are moved in parallel down the strip as shown in the diagram. The third lowermost diagram shows the result of the second electrophoresis, which each of the components separated by the first electrophoresis being separated into further components.

1. A process for assembling molecules on a surface, the process comprising:
providing a probe having a variable-temperature tip;
providing a first material, which is optionally on a first surface of a substrate;
bringing the tip of the probe into contact with the first material, whereby molecules of the first material are transferred to the tip; and
moving the tip of the probe to a position above a surface of a second material (the second surface); then
heating the tip of the probe to a temperature T2, at which the first material will transfer from the tip to the surface of the second material.

2. A process as claimed in claim 1, wherein the molecules of the first material contact the second surface prior to or during the heating of the tip to temperature T2.

3. A process as claimed in claim 1 or, wherein the second surface has reactive moieties on its surface that will form a chemical bond with molecules of the first material.

4. A process as claimed in claim 3, wherein, on contacting molecules of the first material with the reactive moieties, chemical bonds are formed between the first material and the reactive moieties.

5. A process as claimed in claim 4, wherein the species formed from the chemical bonding of a first material with the reactive moieties forms other reactive moieties, and the process as defined in claim 1 is repeated one or more times, wherein the material transferred in the one or more times is the same as or different from the first material.

6. A method for controlling the location of chemical bond formation on a surface, the process comprising:
providing a surface having first reactive moieties associated therewith and second reactive moieties in contact with the first reactive moieties, wherein the second reactive moieties are capable of forming chemical bonds with the first reactive moieties, said surface being at a temperature of below temperature T_{A},
bringing a probe having a temperature of at least T_{A} into contact with the first and second reactive moieties, such that the first reactive moieties form chemical bonds with the second reactive moieties.

7. A method as defined in claim 6, wherein the first and/or second reactive moieties have been transferred to the surface using the method as defined in claim 1 in which the first material comprises the first and/or second reactive moieties.

8. A method as claimed in claim 6, wherein the first reactive moieties comprise species having isocyanate groups and the second reactive moieties comprise polymers having hydroxyl groups.

9. An analytical process comprising:
providing a probe having a tip,
providing a material comprising probe molecules,
bringing the tip of the probe into contact with the material comprising probe molecules, whereby probe molecules are transferred to the tip,
providing a surface having target molecules thereon,
bringing the tip of the probe into close proximity or contact with the target molecules so that the target and probe molecules interact,
and detecting the interactions between the probe molecules and the target molecules.

10. An analytical process as claimed in claim 9, the process further comprising the step of forming the surface having target molecules thereon by one or more of the following:
(i) transferring the target molecules to the surface using the process as defined in claim 1, in which the first material comprises the target molecules;
(ii) forming the target molecules on the surface using the process as defined in claim 5;
(iii) using the method as defined in claim 6 to construct the target molecules on the surface from the first and second reactive moieties.

11. An analytical process comprising:
providing a probe having a tip,
providing a material comprising target molecules,
bringing the tip of the probe into contact with the material comprising target molecules, whereby target molecules are transferred to the tip,
providing a surface having probe molecules thereon,
bringing the tip of the probe into close proximity or contact with the probe molecules so that the target and probe molecules interact,
and detecting the interactions between the probe molecules and the target molecules.

12. An analytical process as claimed in claim 11, the process further comprising the step of forming the surface having probe molecules thereon by one or more of the following:
(i) transferring the probe molecules to the surface using the process as defined in claim 1, in which the first material comprises the probe molecules;
(ii) forming the probe molecules on the surface using the process as defined in claim 5;
(iii) using the method as defined in claim 6 to construct the probe molecules on the surface from the first and second reactive moieties.

13. An analytical process comprising
a) either
i) providing a surface having thereon probe molecules and transferring target molecules to the surface using the process as defined in claim 1 (in which the first material comprises the target molecules), or
ii) providing a surface having thereon target molecules and transferring probe molecules to the surface using the process as defined in claim 1 (in which the first material comprises the probe molecules); and
b) detecting interactions between the target molecules and the probe molecules.

14. An analytical process as defined in claim 13, wherein the interactions between the probe molecules and the target molecules are detected using one or one or more of the following techniques:
a) the surface is imaged by the probe using a contact or intermitted contact or non-contact method and changes due to the bonded target molecules are seen as changes in the corresponding images;
b) scanning the temperature of the tip using a local scanning calorimetry experiment, with or without temperature modulation, combined with thermo mechanical and/or dynamic thermo mechanical analysis to detect a transition temperature or a change in transition temperature;
c) irradiating the tip with electromagnetic radiation at a suitable frequency or frequencies in a photo thermal spectroscopy test;
d) heating the tip to a high temperature causing the molecules on the surface to volatilise or decompose, these volatised and decomposed species then being transported by a flow of air into a mass spectrometer and then analysed.

15. An analytical process comprising:
forming a surface coating on a substrate using a process as defined in claim 1 or a method as defined in claim 6, wherein the surface coating has at least one property which varies in a first direction across the surface,
placing a sample on the surface coating,
and separating the components of the sample along the said direction and detecting and identifying the components of the sample.

16. An analytical process as claimed in claim 15, wherein the pH of the coating varies in the first direction.

17. An analytical process as claimed in claim 15, wherein the components of the sample are separated in the first direction using electrophoresis.

18. An analytical process as claimed in claim 15, wherein, following separation of the components of the sample in the first direction, a further electrophoresis separation is carried out in a second direction on the surface, preferably perpendicular to the first direction.
